(19) United States
(12) Patent Application Publication
(10) Pub. No.: US 2004/0029841 A1
(43) Pub. Date: Feb. 12, 2004

(54) Purinergic antagonists for treating depression

(76) Inventor: Perry F. Renshaw, Bedford, MA (US)

Correspondence Address: CLARK & ELBING LLP, 101 FEDERAL STREET, BOSTON, MA, 02110, US

(21) Appl. No.: 10/413,912

(22) Filed: Apr. 15, 2003

Related U.S. Application Data
(60) Provisional application No. 60/373,459, filed on Apr. 18, 2002.

(51) Int. Cl. A61K 31/7076; A61K 31/655; A61K 31/496; A61K 31/21

(52) U.S. Cl. 514/150; 514/45; 514/253.05; 514/514

(57) ABSTRACT

The present invention provides methods for treating individuals suffering from depression. The methods are based on the administration of a therapeutically effective amount of a substance that lowers the circulating concentration of a purinergic compound or that acts as an antagonist to a purinergic receptor.

### CROSS-REFERENCE TO RELATED APPLICATIONS

This application claims benefit of U.S. Provisional Application No. 60/373,459, filed Apr. 18, 2002, hereby incorporated by reference.

### BACKGROUND OF THE INVENTION

The invention relates to the field of treatment of depression.

Depression is a mental condition that affects millions of people worldwide. Various medications for treating depression are available. These medications belong to several classes of compounds, including tricyclic compounds, selective serotonin reuptake inhibitors (SSRIs), and monoamine oxidase inhibitors. Medications belonging to these classes have numerous potential side effects and are not effective for all individuals suffering from depression.

Thus, there exists a need for new treatments for depression that potentially have fewer side effects or are more effective in treating certain individuals.

### SUMMARY OF THE INVENTION

The present invention provides methods for treating individuals suffering from depression. The methods are based on the administration of a therapeutically effective amount of a substance that lowers the circulating concentration of a purinergic compound or that acts as an antagonist to a purinergic receptor.

In one aspect, the invention features a method for treating an individual suffering from depression. This method includes administering to an individual a therapeutically effective amount of a pharmaceutical composition that contains (i) a compound that acts as an antagonist to a receptor for a purinergic compound or that reduces the circulating concentration of said purinergic compound, excepting S-adenosylmethionine, and (ii) a pharmaceutically acceptable carrier. In various embodiments, the purinergic compound is ATP, ADP, or a combination of ATP and ADP.

Examples of pharmaceutical compositions, according to the invention, include reactive red 2, suramin, 8,8′-(carbonylbis(imino-3,1-phenylene carbonylimino)bis(1,3,5-naphthalenetrisulfonic acid), 8,8′-(carbonylbis(imino-4,1-phenylenecarbonylimino-4,1-phenylene-carbonylimino))bis(1,3,5-naphthalenetrisulfonic acid), Evans blue, trypan blue, reactive blue 2, pyridoxalphosphate-6-azophenyl-29,49-disulfonic acid, isoquinoline sulfonamide, 1-[N,O-bis(5-isoquinoline-sulfonyl)-N-methyl-L-tyrosyl]-4-phenylpiperazine, trinitrophenyl-substituted nucleotides, diinosine pentaphosphate, cicacron blue 3GA, 2′,3′-O-(2,4,6-trinitrophenyl)-ATP, substance P, basilen blue, and 4,4′-diisothiocyanostilbene-2,2′-disulfonic acid, or pharmaceutically acceptable salts thereof.

By “pharmaceutically acceptable salt” is meant a non-toxic salt of a compound of the invention formed, e.g., from non-toxic inorganic or organic acids. Such non-toxic salts include, for example, those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like. Other pharmaceutically acceptable salts are known to those skilled in the art.

By a “derivative” is meant a structural derivative having a chemical modification of the compound that enhances bioavailability, solubility, or stability in vivo or ex vivo, or that reduces the toxicity or dosage required. Such modifications are known to those skilled in the field of medicinal chemistry.

By “treating” is meant the medical management of a patient with the intent that a prevention, cure, stabilization, or amelioration of the symptoms will result. This term includes active treatment, that is, treatment directed specifically toward improvement of the disorder; palliative treatment, that is, treatment designed for the relief of symptoms rather than the curing of the disorder; preventive treatment, that is, treatment directed to prevention of disorder; and supportive treatment, that is, treatment employed to supplement another specific therapy directed toward the improvement of the disorder. The term “treatment” also includes symptomatic treatment, that is, treatment directed toward constitutional symptoms of the disorder.

By “therapeutically effective amount” is meant an amount of a pharmaceutical composition sufficient to produce a preventative, healing, curative, stabilizing, or ameliorative effect either in the treatment of a disorder or in the treatment of symptoms of a disorder, for example, depression.

### BRIEF DESCRIPTION OF THE DRAWINGS

FIG. 1 is a graph depicting the decrease in levels of ATP in healthy individuals after administration of SAMe.

### DETAILED DESCRIPTION OF THE INVENTION

We have discovered that the administration of S-adenosylmethionine (SAMe) to healthy individuals resulted, on average, in the lowering of circulating levels of ATP. This lowering was measured as a decrease in the ratio of beta ATP to total ³¹P MRS metabolites and detected using magnetic resonance imaging (see, e.g., U.S. Pat. No. 6,258,794, hereby incorporated by reference).

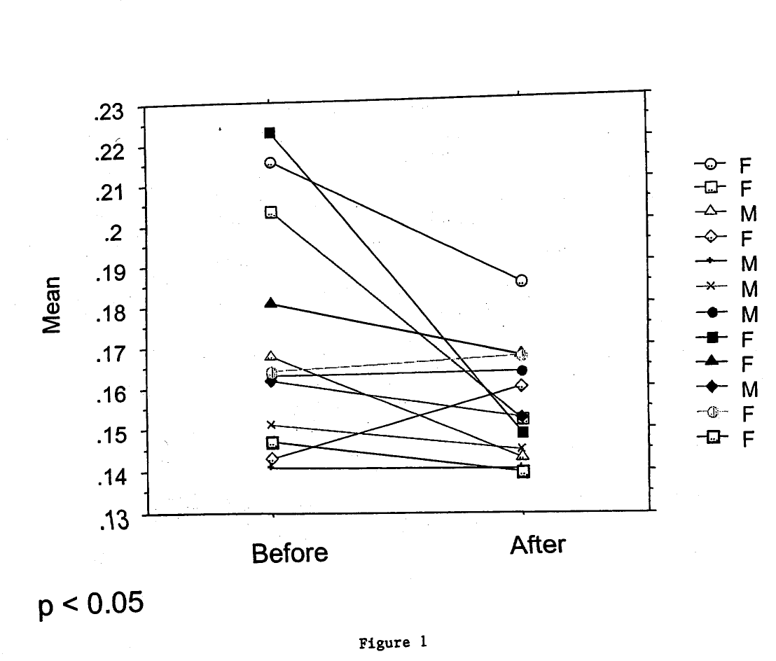

Five healthy men and seven healthy women self-administered 1600 mg of oral SAMe daily. The effect of SAMe on brain bioenergetic status, membrane integrity, and brain transverse relaxation time (T₂) was then examined. In vivo ³¹P magnetic resonance spectra and relaxation times were acquired, at baseline and after 14 and 28 days of treatment, from an axial slice (50 mm) encompassing the bilateral basal ganglia using a 1.5-T scanner. Following SAMe supplementation, mole percent concentrations of phosphocreatine (PCr) were significantly higher than at baseline. In contrast, mole percent beta nucleoside triphosphate (β-NTP) levels, predominantly adenosine triphosphate in the brain, decreased significantly after treatment (see FIG. 1). The changes observed in PCr and β-NTP are consistent with the report that SAMe is involved in the production of creatine, which in turn is phosphorylated to PCr at the expense of β-NTP (Braissant et al., Brain Res. Mol. Brain Res. 2001, 86:193-201).

Women exhibited significantly lower T₂ values than men at the end of the treatment regimen. As changes in T₂ relaxation times are inversely related to alterations in steady state blood flow, the decrease in T₂ observed in women corresponds to a 3.8% increase in cerebral blood flow. These data provide additional support that SAMe facilitates energy utilization in the brain.

SAMe is known to have antidepressant activity, and the above data indicate that SAMe affects the biochemistry of the brain. The neural mechanism underlying this activity, however, is not well understood. As a result of our discovery that SAMe induces reductions in the level of ATP, a neurotransmitter in the human central nervous system that acts at purinergic receptors, it is now possible to administer compounds that reduce the concentration of purinergic compounds or act as antagonists at purinergic receptors to treat depression.

Accordingly, the invention features a method for treating individuals suffering from depression by administering a therapeutically effective amount of a compound that lowers the circulating concentration of a purinergic compound or that acts as an antagonist to a receptor for the purinergic compound combined with a pharmaceutically acceptable carrier, e.g., physiological saline or sterilized water. The purinergic compound is, for example, ADP, ATP, or a combination thereof.

Compounds other than SAMe are known that antagonize purinergic receptors or reduce the circulating concentration of purinergic compounds. These compounds include, for example, reactive red 2, suramin, 8,8′-(carbonylbis(imino-3,1-phenylene carbonylimino)bis(1,3,5-naphthalenetrisulfonic acid) (also called NF023), 8,8′-(carbonylbis(imino-4,1-phenylenecarbonylimino-4,1-phenylenecarbonylimino))bis(1,3,5-naphthalenetrisulfonic acid) (also called NF279), Evans blue, trypan blue, reactive blue 2, pyridoxalphosphate-6-azophenyl-29,49-disulfonic acid (PPADS), isoquinoline sulfonamide, 1-[N,O-bis(5-isoquinoline-sulfonyl)-N-methyl-L-tyrosyl]-4-phenylpiperazine (also called KN-62), trinitrophenyl-substituted nucleotides (e.g., TNP-ATP), diinosine pentaphosphate (IP₅I), cicacron blue 3GA, 2′,3′-O-(2,4,6-trinitrophenyl)-ATP, substance P (SP), basilen blue, and 4,4′-diisothiocyanostilbene-2,2′-disulfonic acid, or combinations, derivatives, or pharmaceutically acceptable salts thereof.

The compounds of the invention can be administered by any standard means for administering therapeutic compounds, including, without limitation, oral, sublingual, transdermal, intravenous, and administration into the cerebrospinal fluid. Dosages, formulations, and timing of administration can be determined using routine methods for such determination.

Purinergic receptors include, for example, any receptors that specifically bind ATP, ADP, or both. In addition, some purinergic receptors bind nucleotide phosphates in addition to ATP and ADP. Examples of purinergic receptors are P2X and P2Y receptors.

The above-described methods can also be used to treat other brain disorders that are potentially characterized by an increase in the level or activity of a purinergic compound. These disorders include, without limitation, bipolar disorder, schizoaffective disorder, dysthymia, cyclothymia, seasonal affective disorder, attention deficit hyperactivity disorder, panic disorder, obsessive-compulsive disorder, and eating disorders (e.g., bulimia and anorexia nervosa).

### Other Embodiments

Modifications and variations of the described methods of the invention will be apparent to those skilled in the art without departing from the scope and spirit of the invention. Although the invention has been described in connection with specific desirable embodiments, it should be understood that the invention as claimed should not be unduly limited to such specific embodiments. Indeed, various modifications of the described modes for carrying out the invention, which are obvious to those skilled in the art, are intended to be within the scope of the invention.

All publications, patents, and patent applications mentioned in this specification are herein incorporated by reference to the same extent as if each individual publication, patent, or patent application was specifically and individually to be incorporated by reference.

Other embodiments are within the claims.

What is claimed is:

1. A method for treating an individual suffering from depression, said method comprising administering to said individual a therapeutically effective amount of a pharmaceutical composition comprising (i) a compound that acts as an antagonist to a receptor for a purinergic compound or a compound that reduces the circulating concentration of said purinergic compound, excepting S-adenosylmethionine, and (ii) a pharmaceutically acceptable carrier.

2. The method of claim 1, wherein said purinergic compound is ATP.

3. The method of claim 1, wherein said purinergic compound is ADP.

4. The method of claim 1, wherein said purinergic compound is ATP or ADP.

5. The method of claim 1, wherein said pharmaceutical composition is selected from the group consisting of reactive red 2, suramin, 8,8′-(carbonylbis(imino-3,1-phenylene carbonylimino)bis(1,3,5-naphthalenetrisulfonic acid), 8,8′-(carbonylbis(imino-4,1-phenylenecarbonylimino-4,1-phenylenecarbonylimino))bis(1,3,5-naphthalenetrisulfonic acid), Evans blue, trypan blue, reactive blue 2, pyridoxalphosphate-6-azophenyl-29,49-disulfonic acid, isoquinoline sulfonamide, 1-[N,O-bis(5-isoquinoline-sulfonyl)-N-methyl-L-tyrosyl]-4-phenylpiperazine, trinitrophenyl-substituted nucleotides, diinosine pentaphosphate, cicacron blue 3GA, 2′,3′-O-(2,4,6-trinitrophenyl)-ATP, substance P, basilen blue, and 4,4′-diisothiocyanostilbene-2,2′-disulfonic acid, or a pharmaceutically acceptable salt thereof.
